(19) United States
(12) Patent Application Publication
(10) Pub. No.: US 2024/0285378 A1
(43) Pub. Date: Aug. 29, 2024

(54) IMPLANT DRILLS, IN PARTICULAR DENTAL IMPLANT DRILLS

(71) Applicant: medentis medical GmbH, Bad Neuenahr-Ahrweiler (DE)

(72) Inventor: Alexander Scholz, Bad Neuenahr-Ahrweiler (DE)

(21) Appl. No.: 18/576,935

(22) PCT Filed: May 23, 2022
(86) PCT No.: PCT/EP2022/063837

§ 371 Date: Jan. 5, 2024

(30) Foreign Application Priority Data
Jul. 5, 2021 (DE) 10 2021 117 272.1

(51) Int. Cl. A61C 8/00

(52) U.S. Cl. CPC A61C 8/0089

(57) ABSTRACT

An implant drill includes a mounting portion and a treatment portion, wherein the implant drill extends in a longitudinal direction, wherein distal end regions in the longitudinal direction of the implant drill are formed by the mounting portion and the treatment portion, wherein the treatment portion has at least two treatment structures, wherein the treatment portion is formed by the treatment structures such that the treatment portion is configured to remove bone material during a positive rotation about the longitudinal direction and to compact bone material during a negative rotation about the longitudinal direction.

### BACKGROUND

The invention relates to an implant drill, in particular a dental implant drill.

Implants are already known from the prior art. These usually bear a prosthesis and are firmly anchored in the surrounding bone. In order to insert an implant into the bone, a recess must be created in the bone. Depending on the surrounding bone, a decision must be made as to whether the recess in the bone is to be created by drilling or compacting. In the prior art, these two activities are achieved using different tools, namely a drill or a set of drills on the one hand and one or several compaction tools, such as a chisel, on the other. The preparation of different tools can lead to confusion as well as a high workload.

It is therefore the object of the present invention to provide a simple and reliable way of preparing a recess for an implant in a bone, such as a jawbone, in particular of a human being.

### SUMMARY

According to the present invention, an implant drill, in particular a dental implant drill, is provided. For practical purposes, the implant drill comprises a mounting portion and a treatment portion, which can be formed as a drilling portion, wherein the implant drill extends, in particular in or along a longitudinal direction, wherein the distal end regions in the longitudinal direction of the implant drill are formed in particular by the mounting portion and the treatment portion, wherein the treatment portion can have at least two treatment structures, which can be formed as drilling structures, wherein the treatment portion is formed, in particular by the treatment structures, in such a way that it can remove bone material during a positive rotation about the longitudinal direction and that it can compact bone material during a negative rotation about the longitudinal direction. The implant drill is used to create a recess or hole in a bone, in particular a human jaw, by means of a rotational movement. This hole is used to accommodate an implant, in particular a dental implant. The implant drill comprises a mounting portion which is designed or serves to secure the implant drill in a machine tool or a drilling machine or a handpiece. This securing is in particular such that a rotational movement, in particular form-fitting, can be transmitted to the implant drill and/or that the implant drill can be held in the direction of the longitudinal direction, in particular in a form-fitting way. For practical purposes, the mounting portion of the implant drill is advantageously provided with form-fitting structures that allow a torque to be transmitted in a form-fitting way about the longitudinal direction and/or a holding force to be transmitted along the longitudinal direction. For example, this can be achieved by a circumferential groove and/or a sectional surface, which can also be described as a flattening, for example. In addition to the mounting portion, the implant drill also has a treatment portion. This treatment portion is in particular the area of the implant drill that comes into direct contact with the bone material during drilling in order to reach or create the recess or hole in the jaw or bone material. In other words, the treatment portion of the implant drill is used to create the hole. The implant drill extends in particular in a longitudinal direction, wherein the longitudinal direction, for practical purposes, is the direction in which the implant drill rotates during drilling or about which the implant drill rotates. Alternatively or additionally preferably, the longitudinal direction can also be the direction in which the implant drill has its largest main dimension. For practical purposes, one of the distal end areas is formed in the longitudinal direction of the implant drill by the mounting portion and/or, in particular opposite, by the treatment portion. Advantageously, the positive longitudinal direction points from the mounting portion to the treatment portion or vice versa. By designing the treatment portion as the distal end area of the implant drill in the longitudinal direction, a particularly simple drilling option can be created using the implant drill. By forming a distal end region, in particular an opposite one, in the longitudinal direction through the mounting portion, it is possible to achieve particularly simple mounting of the implant drill in a tool holder, in particular a drill, a milling cutter or a machine tool or a hand tool. The treatment portion of the implant drill is advantageously equipped with at least two treatment structures. These treatment structures are in particular those surfaces, edges or other structures of the treatment portion that cause bone reshaping and/or bone separation, especially when the drill rotates. In other words, the flat or linear treatment structures can cause a reshaping and/or removal of bone material, wherein this drilling or change in shape can be or is achieved by implant drill's rotation about the longitudinal direction. The treatment portion is designed, in particular by the treatment structures, in such a way that it can remove bone material during a positive rotation about the longitudinal direction and that it can compact bone material during a negative rotation about the longitudinal direction. In other words, the treatment portion of the implant drill can be designed in such a way that bone material is removed when the implant drill is rotated about the longitudinal direction in one direction, as is the case with a milling cutter or drill, for example. However, if the drill rotates in the opposite direction about the longitudinal direction, the treatment portion is designed in such a way that it causes compaction of the bone material to be drilled, in particular through direct contact of inclined surfaces with the bone material. This design of the different drilling options of the implant drill enables both the removal of bone material and the compaction of the bone material to be achieved with a single tool. For practical purposes, the implant drill or the treatment portion, in particular the treatment structures, is designed in such a way that it can only remove bone material during a positive rotation about the longitudinal direction and that it can only compact bone material during a negative rotation about the longitudinal direction. In other words, the implant drill can be designed in such a way that during a rotation about the longitudinal direction, for example during a positive rotation about the longitudinal direction, only bone material is removed, in particular by exclusive contact of geometrically defined cutting edges with the bone material, and that during an opposite rotation about the longitudinal direction, only bone-compaction treatment structures come into contact with the bone, so that only bone compaction takes place during such a rotation.

Advantageously, the treatment portion has at least two, preferably at least three, spiral grooves, with the spiral grooves at least partially forming and/or bounding the treatment structures or some treatment structures. A spiral groove is a groove that spirals about the longitudinal direction of the implant drill. By introducing a spiral groove, a particularly compact design of the treatment structure can be achieved, especially for bore treatment structures. In addition, the spiral grooves can also be used in a simple manner to provide a space for receiving bone material that is removed. By providing at least two, preferably at least three spiral grooves, a high cooling effect can be achieved and, in addition, this type of design can also achieve a particularly homogeneous intervention characteristic of the treatment portion with bone material during drilling so that the vibrations caused by the implant drill during drilling of the bone material can be reduced.

For practical purposes, the spiral groove at least partially forms and/or bounds the treatment structures or some treatment structures, in particular in a circumferential direction. In particular, this circumferential direction is formed circumferentially about the longitudinal direction. By utilising the spiral grooves in such a way that they partially form and/or bound the treatment structures, a particularly compact design of the implant drill can be achieved.

Advantageously, the treatment portion has different treatment sections along the longitudinal direction, the implant drill preferably having at least three treatment sections, wherein the treatment sections form different outer contour pitch angles with the longitudinal direction or at least one of the treatment sections, preferably all of them, being cylindrical about the longitudinal direction. An outer contour pitch angle is understood to be the angle which a cone has which can only just surround the treatment section and wherein the axis of rotation symmetry of this cone is in the longitudinal direction. Advantageously, the outer contour pitch angle is constant over the respective treatment section. This makes it particularly easy to drill or produce the respective treatment section. A cylindrical design about the longitudinal direction of the treatment sections means in particular that the outer contour of the cone, which directly surrounds the treatment section and whose axis of rotation symmetry is on the longitudinal direction, can only intersect the longitudinal direction itself at infinity. In other words, the outer contour is not enclosable or enclosed by a cone but by a cylinder. The advantage of a cylindrical design is that it is particularly easy to manufacture. The advantage of the different angles of the various treatment sections, which can also be referred to as drilling sectors, is that this enables the drill to create the implant recess in the bone material without the need to change tools. The implant drill can therefore be designed in such a way that, with the exception of possible pre-drilling, the recess in the bone for the implant can be made exclusively with the one implant drill. As a result, time-consuming tool changes can be saved and tool mix-ups can be prevented or at least reduced. For practical purposes, the implant drill is designed in such a way that treatment sections with an outer contour pitch angle and treatment sections with a cylindrical design are arranged alternately in the longitudinal direction. For practical purposes, for each treatment section (in itself) to be designed in such a way that it can remove bone material during a positive rotation about the longitudinal direction and that it can compact bone material during a negative rotation about the longitudinal direction. In other words, the treatment sections can differ from one another solely in terms of their geometric design, but can be designed to be equivalent in terms of their functionality.

Advantageously, the implant drill, in particular the treatment portion, is limited by an axial treatment section in the longitudinal direction, wherein the axial treatment section forms an outer contour pitch angle with the longitudinal direction, wherein the outer contour pitch angle of the axial treatment section is in a range from 60° to 87° and/or wherein the length of the axial treatment section in the longitudinal direction is in a range from 0.2 mm to 0.4 mm. By providing an axial treatment section the core task of which is axial penetration of bone material, a particularly good possibility of penetration of the jaw bone can be achieved if this bounds the treatment portion in the longitudinal direction. If the axial treatment section forms an outer contour pitch angle with the longitudinal direction, which is in the range of 60° to 87°, this can be used to provide a particularly fast and yet effective axial penetration of the implant drill into the bone material. If the length of the axial treatment section in the longitudinal direction is in the range of 0.2 mm to 0.4 mm, a particularly compact design of the axial treatment section can be achieved, so that a particularly easy-to-handle implant drill can be achieved, wherein this can be particularly decisive for dental implant drills, because the handling area for a drill is particularly small in the dental field.

For practical purposes, the implant drill, in particular the treatment portion, has a main treatment section, wherein the main treatment section forms an outer contour pitch angle with the longitudinal direction, wherein the outer contour pitch angle of the main treatment section is in a range of 0.2° to 9°, or wherein the main treatment section is formed cylindrically about the longitudinal direction. The main treatment section is in particular the treatment section of the implant drill that has the highest or greatest length in the longitudinal direction. The main treatment section is primarily used to ensure the final shape of the implant hole or bone recess that is to be created by the implant drill. In particular, the main treatment section therefore preferably forms a distal end of the bone recess. If the main treatment section forms an outer contour inclination angle with the longitudinal direction, wherein this can preferably be in a range of 0.2° to 9°, this can achieve particularly simple and good mountability, in particular of self-tapping implants, additionally or alternatively preferably, dental implants. However, if the main treatment section is cylindrical about the longitudinal direction, this makes it particularly easy to manufacture the drill and also achieves a good retention force for the implant in the bone.

Advantageously, the implant drill, in particular the treatment portion, especially preferably the main treatment section, has circumferential cooling grooves, wherein the cooling grooves form closed rings around the longitudinal direction in particular. By providing grooves or cooling grooves, on the one hand the available surface area is increased and, on the other hand, the cutting surface of the cutting treatment structures, in particular the bore treatment structures, can be reduced, wherein both these effects can contribute to cooling. Providing sufficient cooling is fundamentally important in order to prevent overheating-related trauma to the bone material being processed. In particular, the cooling grooves run in closed rings about the longitudinal direction. A closed ring means that the cooling grooves or the virtual extension of the cooling grooves is closed in itself and is advantageously circular in shape. In other words, the cooling groove can therefore be interrupted along its course, for example by spiral grooves or by treatment structures, wherein the cooling groove is continued behind this interruption and at least the cooling groove course forms a self-contained structure, in particular a circular self-contained structure, complementing the virtual course. The closed rings of the cooling groove make production particularly quick and easy. Ideally, the extension plane of these closed rings is perpendicular to the longitudinal direction. This further simplifies the production of the cooling grooves. Alternatively or additionally preferably, the centre point around which the cooling groove or cooling grooves are formed can be in the longitudinal direction in order to achieve fast, simple and precise production.

For practical purposes, the implant drill, in particular the treatment portion, has a cylindrical treatment section, wherein the cylindrical treatment section is cylindrical about the longitudinal direction. For practical purposes, the cylinder treatment section is formed in the longitudinal direction between the axial treatment section and the main treatment section. It is particularly useful if the cylinder treatment section is formed in the longitudinal direction adjacent to the axial treatment section. The cylindrical design of the cylinder treatment section enables particularly good radial guidance of the drill. This guiding effect of the cylinder treatment section can be increased by the cylinder treatment section being arranged directly adjacent to the axial treatment section in the longitudinal direction and/or at least in the longitudinal direction between the axial treatment section and the main treatment section.

Preferably, the implant drill, in particular the treatment portion, has a transition treatment section, wherein the transition treatment section forms an outer contour pitch angle with the longitudinal direction, wherein the outer contour pitch angle of the transfer section is in particular in a range from 7° to 15°. By providing a transition treatment section, it is particularly easy to increase the diameter of the treatment portion. The transition treatment section is located in the longitudinal direction, in particular between the axial treatment section and the main treatment section. For practical purposes, the transition treatment section forms that section of the treatment portion which is formed in the longitudinal direction between the cylinder treatment section and the main treatment section. By forming the transition treatment section with an outer contour pitch angle in a range of 7º to 15°, particularly low heat development can be achieved while the bone material is being drilled, so that overheating-related bone trauma can be avoided or at least reduced.

Advantageously, the ratio of the diameter of the cylinder treatment section to the diameter of the main treatment section is in a range from 0.5 to 0.9, preferably in a range from 0.72 to 0.82. The diameter of the cylinder treatment section is the diameter of the cylinder directly surrounding the cylinder treatment section, wherein this virtual cylinder has an axis of rotational symmetry which is congruent with the longitudinal direction. In other words, the diameter of the cylinder treatment section is the diameter of the circle which can directly surround the cylinder treatment section or the main treatment section in a sectional plane perpendicular to the longitudinal direction, and the centre of this circle is in the longitudinal direction. This smallest possible circle in the sectional plane is also referred to as an envelopment in the sense of the invention. With a ratio in the range of 0.5 to 0.9, a particularly simple production of the implant drill can be achieved. However, if the ratio is in the range of 0.72 to 0.82, a particularly good guiding effect can be achieved by the cylinder treatment section.

For practical purposes, the ratio of the length of the cylinder treatment section in the longitudinal direction to the length of the main treatment section in the longitudinal direction is in a range from 0.1 to 0.4, preferably in a range from 0.2 to 0.3. With a ratio in the range of 0.1 to 0.4, a particularly good guiding effect of the cylinder treatment section can be provided-even when the bone material is compacted. However, if the ratio is in the range of 0.2 to 0.3, the applicant has surprisingly discovered that this can create a recess which can achieve a particularly good implant stability.

Advantageously, the treatment portion has at least one compaction treatment structure and one bore treatment structure. In particular, a treatment structure is a treatment structure that has a geometrically defined cutting edge. A geometrically defined cutting edge is used in particular to achieve drilling of bone material by chipping. Such a bore treatment structure can be achieved, for example, by undercutting two (boundary) surfaces that come into contact with each other. In other words, the bore treatment structure can in particular be the cutting edge of two merging surfaces, wherein these surfaces can in particular form an undercut with each other. A compaction treatment structure is in particular a structure intended for drilling bone, which can be formed, for example, by a trailing surface that can exert pressure on the bone in order to displace it, in particular in the direction normal to the wall of the hole to be created, and thus achieve compaction of the bone material. For example, such a compaction treatment structure can be achieved by a free-form surface or a free surface, which is designed in such a way that it causes bone compaction through rotation or locally pushes or displaces bone material that is in contact with the surface outwards. This can be achieved, for example, by the free-form surface or the free surface forming an angle and thereby displacing the bone material outwards. In particular, this free-form surface or free surface can be designed in such a way that it is a surface that rises in the direction of rotation, which is designed in particular with a negative rotation about the longitudinal direction in such a way that the free surface forms an increasing distance from the axis of rotation or the longitudinal direction. In other words, this free surface or free-form surface can therefore be designed without undercuts so that it does not cause bone material to be separated during rotation, in particular during negative rotation about the longitudinal direction. By providing different treatment structures, namely compaction treatment structures and bore treatment structures, the treatment portion and its treatment structures can be produced in a simple manner and on the one hand cause compaction and bone removal.

Advantageously, at least one compaction treatment structure, preferably all compaction treatment structures, is formed by a free surface and/or wherein the compaction treatment structure or compaction treatment structures forms or form an angle in a range of 10° to 45°, preferably in a range of 12° to 40°, with a tangent in a cutting plane perpendicular to the longitudinal direction. The relevant tangent is the tangent at the/an envelopment of the treatment portion at which the—albeit virtually extended-course of the compaction treatment structure intersects the envelopment of the treatment portion of the implant drill. The envelopment of the implant drill or the treatment portion is the smallest possible circle whose centre lies in the longitudinal direction and which can directly surround the implant drill or the treatment portion in the cutting plane. In other words, the envelopment can therefore also be formed by a circle that lies in a cutting plane perpendicular to the longitudinal direction, the circle being the smallest possible circle that can surround the treatment portion in the cutting plane. If the angle of the compaction treatment structure is in the range of 10° to 45°, this can result in a compaction treatment structure that is particularly easy to produce. However, if the angle is in the range of 12º to 40°, a particularly good bone compaction option can be achieved, which nevertheless prevents the bone material from overheating during compaction and/or at least greatly reduces the probability of this occurring.

For practical purposes, the compaction treatment structure is designed without undercuts with the directly neighbouring structures, in particular in a cutting plane perpendicular to the longitudinal direction. This undercut-free design with neighbouring structures can, in particular, prevent bone material from being separated during rotation about the longitudinal direction by the compaction treatment structure or the compaction treatment structures.

Advantageously, the angle of the compaction treatment structure is variable along the course in the longitudinal direction. In other words, the angle formed by the compaction treatment structure in different cutting planes along the longitudinal direction can therefore be different. In particular, this allows the compaction effect of the compaction treatment structure to be adapted to the outer diameter of the drill. This is particularly advantageous due to the fact that different diameters require different angles of approach for bone compaction. For practical purposes, however, the angle of the compaction treatment structures along the course in the longitudinal direction is constant in the respective treatment sections. This can simplify the production of the drill. In other words, the angle can, for example, be constant in the main treatment section and/or in every other treatment section. However, the angle can change at the transition from one treatment section to another, so that it is not “globally” constant but variable, especially along the longitudinal direction.

Advantageously, the angle of the compaction treatment structure, preferably of all compaction treatment structures, in the axial treatment section and/or in the cylinder treatment section is in a range of 12° to 18°. In this way, a particularly good compaction effect can be achieved with small diameters of the drill, whereby overheating of the bone material can still be avoided or at least greatly reduced.

Advantageously, the angle of the compaction treatment structure, preferably of all compaction treatment structures, in the main treatment section and/or in the transition treatment section is in a range of 30° to 40°. An angle in the range of 30 to 40°, particularly preferably in the range of 33° to 37°, has a particularly good compacting effect, especially with large drilling areas or treatment structures, which primarily do not cause axial drilling, wherein bone overheating can be avoided and/or at least reduced at the same time.

Advantageously, the angle of the compaction treatment structure, preferably of all compaction treatment structures, is predominantly constant. A predominantly constant angle means that the angle only changes in small sections in the longitudinal direction. In other words, although the angle of the compaction treatment structure may be variable when viewed along the longitudinal direction, the sum of the constant sections is 70%, preferably at least 80% and particularly preferably at least 90% of the length of the compaction treatment structure in the longitudinal direction so as to be considered predominantly constant within the meaning of the invention. For example, with the exception of a sharp transition, the angle in the axial treatment section and/or in the cylinder treatment section can assume a first value and a second value in the main treatment section and/or in the transition treatment section. These different angle ranges are limited in particular by a sharp transition area or this sharp transition area itself has a variable angle. The sharp transition area can preferably be designed as an edge, resulting in an abrupt transition. Alternatively, the transition area can also preferably extend in a length range of 0.01 to 0.4 mm, preferably in a range of 0.02 to 0.1 mm, in the longitudinal direction. In other words, except for the transition area, the angle can be constant in each section.

Preferably, the spiral grooves, preferably all spiral grooves, are bounded in the circumferential direction by a compaction treatment structure and/or by a bore treatment structure and/or by a transition chamfer. In other words, the spiral groove, in particular in the circumferential direction around the longitudinal direction, can be bounded in the positive and/or negative circumferential direction around the longitudinal direction by a compaction treatment structure, a bore treatment structure and/or a transition chamfer in each case. This enables a small size of the implant drill to be achieved. A transition chamfer is a short chamfer, in particular with an edge length in the range of 0.02 to 0.1 mm, which provides a transition to a compaction treatment structure. This transition chamfer can prevent undercutting in particular, so that it can be ensured that the compaction treatment structure and the area surrounding it do not have a bone-eroding effect.

For practical purposes, at least one spiral groove, preferably all spiral grooves, extends through the main treatment section, the transition treatment section, the cylinder treatment section and/or through the axial treatment section. By designing the spiral groove in such a way that it runs through several treatment sections, in particular all treatment sections, in the longitudinal direction and/or extends into these areas, a particularly simple production of the implant drill can be achieved. If the spiral groove extends through or into all areas of the treatment section, a particularly good possibility can be created for the drill to safely prepare/create all areas of the hole or implant recess not only during machining processes but also during compaction drilling, and at the same time the spiral groove can also provide a cooling effect for the bone material.

Advantageously, the treatment portion is bounded in the longitudinal direction by a stop flange, in particular one that is circumferential in the longitudinal direction. This ensures that the implant drill is prevented from penetrating too deeply in a form-fitting way into the bone material. A stop flange of this type can therefore increase the safety of the implant drill.

Advantageously, the implant drill is made of titanium or a titanium alloy. The use of titanium can achieve particularly good stability of the drill and also a particularly high mechanical load capacity of the drill, so that its safety can be increased and the duration of use of the implant drill can be positively influenced.

The implant drill is conveniently designed as a single piece. The term “single piece” means in particular that the relevant component is fundamentally manufactured from a body that was created in a single original moulding process. In other words, it can no longer be a single-piece design if different components have to be joined to create an initial body or blank from which the implant drill was created. Due to the single-piece design of the implant drill, a particularly good strength of the drill can be achieved, so that it is particularly advantageous, especially with regard to a dynamic and/or pulsating load.

Advantageously, the implant drill is designed as a solid part. A solid part means in particular that the implant drill is not hollow. In other words, the implant drill can be a solid part that has no holes or openings. This can have a positive effect on mechanical stability in particular.

### BRIEF DESCRIPTION OF THE DRAWINGS

Further advantages and features of the present invention are shown in the following description with reference to the figures. Individual features of the embodiments shown can also be used in other embodiments, unless this has been expressly excluded. This includes:

FIG. 1 is a side view of an implant drill;

FIG. 2 is a detailed view of an implant drill in the area of the treatment portion;

FIG. 3 is a cross-section through a treatment portion of the implant drill;

FIG. 4 is another cross-section through the treatment portion of an implant drill;

FIG. 5 is an isometric view of an implant drill;

FIG. 6 is a side view of an implant drill blank;

FIG. 7 is an axial view of an implant drill; and

FIG. 8 is a schematic view of a cross-section of a treatment portion of the implant drill with an envelopment.

### DETAILED DESCRIPTION OF THE PREFERRED EMBODIMENTS

FIG. 1 shows a side view of an implant drill 1. The distal ends in the longitudinal direction L of the implant drill 1 are formed by the mounting portion 10 and the treatment portion 30. In the axial direction or in the longitudinal direction L, the treatment portion 30 is bounded by the axial treatment section B1 and by the stop flange 60. The treatment portion 30 can be broken down into or is formed by four sections which are arranged side by side in the longitudinal direction, these treatment sections being the axial treatment section B1, the cylindrical treatment section B2, the transition treatment section B3 and the main treatment section B4. The treatment portion 30 has multiple spiral grooves 34, which extend spirally about the longitudinal direction L and extend from the axial treatment section B1, via the cylinder treatment section B2, via the transition treatment section B3 and into the main treatment section B4. In order to achieve a particularly good cooling effect drilling into bone, the main treatment section B4 has multiple cooling grooves 36, which are formed in a ring about the longitudinal direction L. In order to secure the implant drill 1 in a drill or a hand tool, the mounting portion 10 has a flattened section and a circumferential groove to enable positive torque transmission about the longitudinal direction to the implant drill 1 and positive axial force transmission in the direction of the longitudinal direction L. The main treatment section B4 can extend as far as the stop flange 60 in the longitudinal direction L.

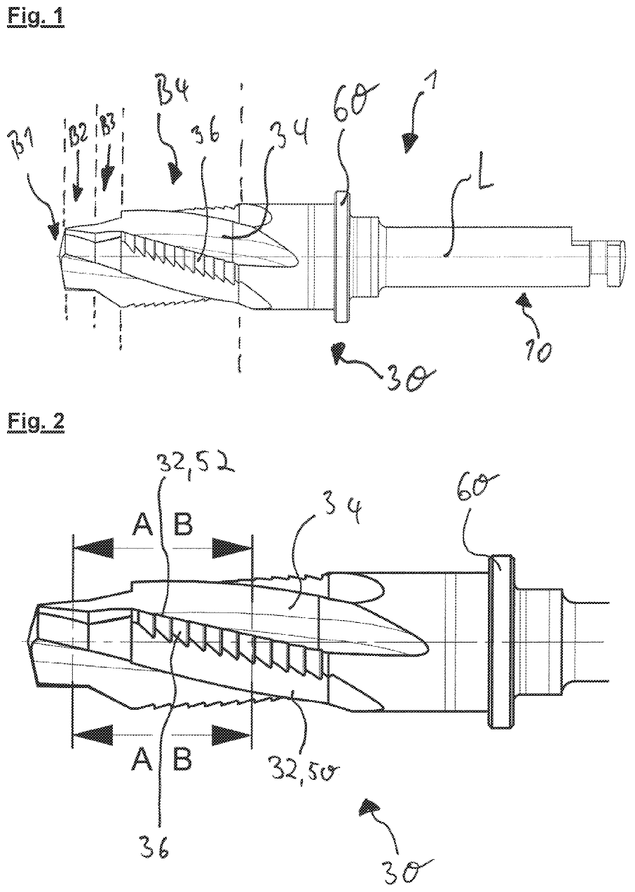

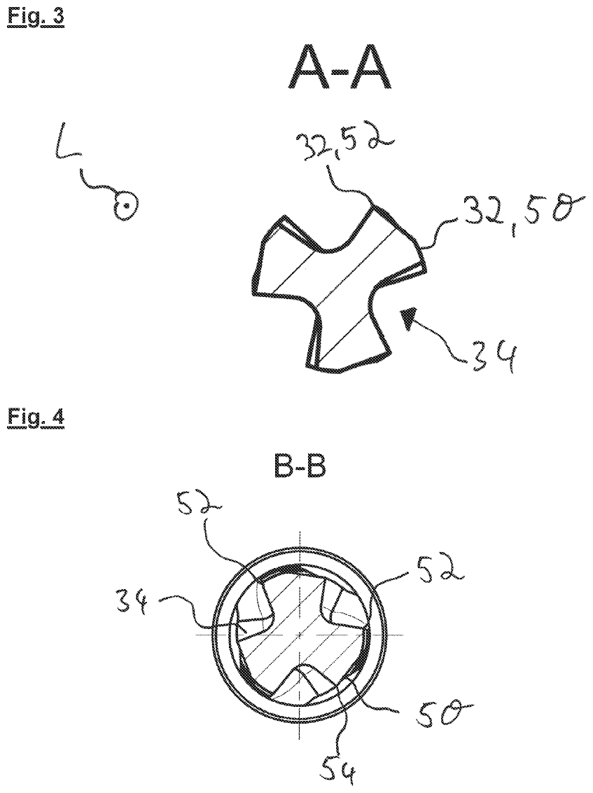

FIG. 2 shows a detailed view of a treatment portion 30. The treatment portion 30 shown in FIG. 2 can correspond in particular to the treatment portion 30 shown in FIG. 1. FIG. 2 shows two sectional planes marked with arrows, labelled A and B. Possible configurations of these sectional planes can be found for the sectional plane A-A in FIG. 3 and B-B in FIG. 4. The treatment portion 30 has multiple spiral grooves 34. These spiral grooves 34 are bounded in the circumferential direction around the longitudinal direction L by treatment structures 32. The spiral groove 34 is bounded on one side in the circumferential direction by bore treatment structures 52 and in the opposite circumferential direction by compaction treatment structures 50. However, the compaction treatment structures 50 can be spaced from the spiral groove 34 by a transition chamfer 54. In other words, a short transition chamfer 54 can be provided on the output side of the spiral groove 34, into which the compaction treatment structure 50 then joins when viewed in the circumferential direction. The axial run-out of the spiral groove 34 is located in the main treatment section B4 as viewed in the longitudinal direction L.

FIG. 3 shows a possible sectional view of the sectional plane labelled A-A in FIG. 2. As can be seen in FIG. 3, the implant drill 1 has three spiral grooves 34 arranged equidistantly in the circumferential direction. The spiral grooves 34 are formed at the outer intersection with the envelopment in such a way that they form either a compaction treatment structure 50 or a bore treatment structure 52. These two types of treatment structures 32 are used to create a recess or hole in bone material to enable the placement of an implant within this bone material.

FIG. 4 shows a possible configuration of the section shown as B-B in FIG. 2.

The section B-B preferably runs through the main treatment section B4. In the main treatment section B4, the spiral grooves 34 are also bounded by bore treatment structures 32 and/or compaction treatment structures 40 and/or transition phases 54 in a circumferential direction about the longitudinal direction L. In other words, the distal end regions of the spiral groove 34 may form the treatment structures 32 and/or a transition phase 54.

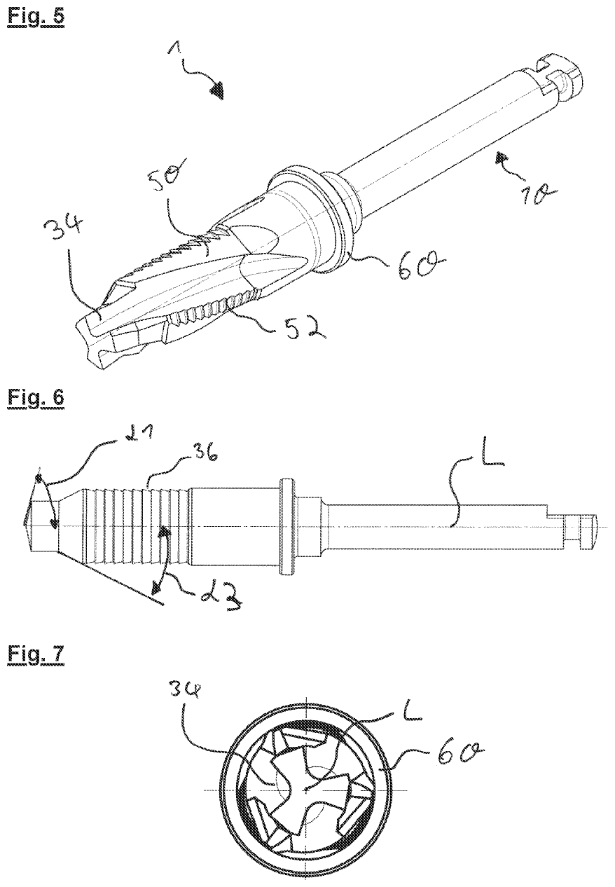

FIG. 5 shows an explanatory isometric view of an implant drill 1. FIG. 5 clearly shows the flattened torque transmission surface in the mounting portion 10 of the implant drill 1. As can be seen from FIG. 5, the spiral grooves 34 extend from one axial end in the longitudinal direction L of the treatment portion 30 over large parts of the treatment portion 30.

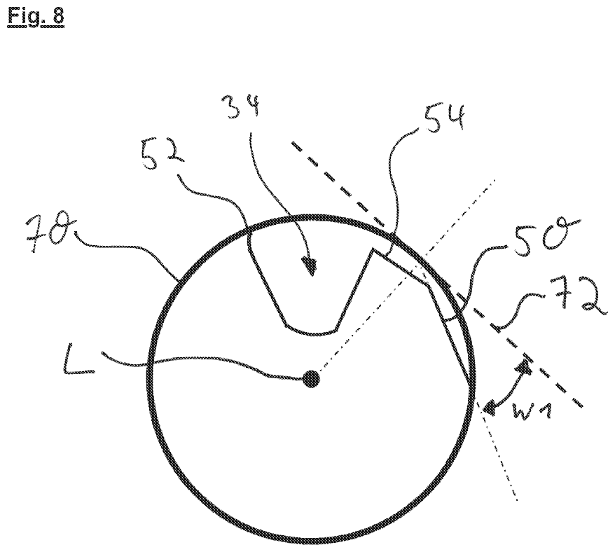

FIG. 6 shows a blank or semi-finished product of an implant drill 1. FIG. 6 illustrates the outer contour pitch angle α1 of the axial treatment section B1 and the outer contour pitch angle α3 of the transition treatment section B3. No spiral grooves 34 are provided in the implant drill 1 or the implant drill blank shown in FIG. 6, with the result that the implant drill 1 or the implant drill blank-as shown in FIG. 6—is formed without treatment structures 32. However, as can be seen in FIG. 6, the cooling grooves 36 are already included in the treatment section 30 of the blank, which are formed in closed rings around the longitudinal direction L in the shape of a circle. As can be seen from FIG. 6—in comparison to FIG. 1—the cylinder treatment section B2 and the main treatment section B4 are cylindrical about the longitudinal direction L. In principle, the implant drill 1 shown in FIG. 6 or the implant drill blank can be used as an initial workpiece for the implant drills shown in FIGS. 1, 2, 3, 4, 5 and 7 and also in principle for the embodiment example shown in FIG. 8. In other words, an implant drill according to FIGS. 1 to 5 and 7 to 8 can be formed from the blank according to FIG. 6.

FIG. 7 shows a view of an implant drill 1 in the longitudinal direction L. The positive longitudinal direction L can point from the mounting portion 10 to the treatment portion 30 or from the treatment portion 30 to the mounting portion 10. Advantageously, the implant drill 1 is designed in such a way that it achieves bore drilling when turned clockwise and compaction drilling when turned anti-clockwise. Such an embodiment can be achieved, for example, by the embodiment shown in FIG. 7.

FIG. 8 shows an illustration, albeit only schematic, to explain the principle of an embodiment of a compaction treatment structure 50. FIG. 8 shows a sectional plane perpendicular to the longitudinal direction L, wherein this sectional plane is located within the treatment portion 30. Piercing the cutting plane in the longitudinal direction L, a spiral groove 34 is provided in the implant drill 1, which is bounded by a treatment structure 52 in an anti-clockwise circumferential direction. A transition chamfer 54 adjoins the spiral groove 34 in a clockwise circumferential direction, which is followed by a compaction treatment structure 50 in a clockwise circumferential direction. The treatment portion 30 is surrounded by the envelopment 70, whereby the centre of this “virtual” envelopment 70 is in the longitudinal direction L. The at least “virtual” course of the compaction treatment structure 50 intersects the envelopment 70 in the sectional plane shown. The tangent 72 applied to the envelopment 70 at this intersection point has an angle W1 with the compaction treatment structure 50 or the virtual course of the compaction treatment structure 50.

### LIST OF REFERENCE SYMBOLS

1—Implant drill
10—Mounting portion
30—Treatment portion
32—Treatment structures
34—Spiral groove
36—Cooling groove
50—Compaction treatment structure
52—Bore treatment structure
54—Transition chamfer
60—Stop flange
70—Envelopment
72—Tangent
B1—Axial treatment section
B2—Cylinder treatment section
B3—Transition treatment section
B4—Main treatment section
L—Longitudinal direction
W1—Angle of the compaction treatment structure
a1—Outer contour pitch angle of the axial treatment section
a3—Outer contour pitch angle of the transition treatment section

1.-15. (canceled)

16. A dental implant drill, comprising:
a mounting portion and a treatment portion;
wherein the implant drill extends in a longitudinal direction;
wherein distal end regions of the implant drill in the longitudinal direction of the implant drill are formed by the mounting portion and the treatment portion;
wherein the treatment portion has at least two treatment structures;
wherein the treatment portion is formed by the at least two treatment structures such that the treatment portion is configured to remove bone material during positive rotation about the longitudinal direction and to compact bone material during a negative rotation about the longitudinal direction;
wherein the treatment portion has at least one compaction treatment structure and one bore treatment structure;
wherein an envelopment is formed by a circle which lies in a sectional plane which is perpendicular to the longitudinal direction;
wherein the circle is the smallest possible circle that can surround the drilling portion in the sectional plane;
wherein an at least virtual course of the compaction treatment structure intersects the envelopment in the sectional plane at a point of intersection, and wherein a tangent applied to the envelopment at the point of intersection has an angle with the compaction treatment structure or the virtual course of the compaction treatment structure;
wherein the angle is in a range from 10° to 45°; and
wherein the treatment portion has circumferential cooling grooves.

17. The implant drill according to claim 16, wherein the angle is in a range of from 12° to 40°.

18. The implant drill according to claim 16, wherein the treatment portion has at least two spiral grooves, and wherein the spiral grooves at least partially form and/or bound at least some of the treatment structures.

19. The implant drill according to claim 18, wherein the treatment portion has at least three spiral grooves.

20. The implant drill according to claim 16, wherein the at least two treatment sections include at least three treatment sections that form different outer contour pitch angles with the longitudinal direction or are formed cylindrically about the longitudinal direction.

21. The implant drill according to claim 16, wherein the treatment portion is bounded by an axial treatment section in the longitudinal direction, wherein the axial treatment section forms an outer contour pitch angle with the longitudinal direction, wherein the outer contour pitch angle of the axial treatment section is in a range from 60° to 87° and/or wherein the length of the axial treatment section in the longitudinal direction is in a range from 0.2 mm to 0.4 mm.

22. The implant drill according to claim 16, wherein the cooling grooves form closed rings about the longitudinal direction.

23. The implant drill according to claim 16, wherein the treatment portion has a cylinder treatment section, and wherein the cylinder treatment section is cylindrical about the longitudinal direction.

24. The implant drill according to claim 16, wherein the treatment portion has a transition treatment section that forms an outer contour pitch angle with the longitudinal direction, and wherein the outer contour pitch angle of the transition treatment section is in a range from 7° to 15°.

25. The implant drill according to claim 16, wherein the at least one compaction treatment structure is formed by a free surface.

26. The implant drill according to claim 25, wherein the at least one compaction treatment structure includes a plurality of compact drill structures, and wherein all of the compact drill structures are formed by the free surface.

27. The implant drill according to claim 16, wherein the angle of the compaction treatment structure is variable along the course in the longitudinal direction.

28. The implant drill according to claim 16, wherein the treatment portion includes a plurality of spiral grooves that are bounded in the circumferential direction by a compaction treatment structure and/or by a bore treatment structure or a transition chamfer.

29. The implant drill according to claim 16, wherein the treatment portion includes at least one spiral groove that extends through the main machining section, the transition machining section, the cylinder machining section and/or through the axial machining section.

30. The implant drill according to claim 16, wherein the treatment portion is bounded in the longitudinal direction by a stop flange.

31. The implant drill according to claim 16, whereby the implant drill is a single piece.

32. The implant drill according to claim 16, whereby the implant drill is configured as a solid part.
